(19) United States
(12) Patent Application Publication
(10) Pub. No.: US 2012/0077221 A1
(43) Pub. Date: Mar. 29, 2012

(54) DETECTION DEVICE AND METHOD FOR DETECTING ANALYTE

(75) Inventor: Scott F. GRIMSHAW, Greenville, SC (US)

(73) Assignee: Tangidyne LLC, Greenville, SC (US)

(21) Appl. No.: 13/312,102

(22) Filed: Dec. 6, 2011

Related U.S. Application Data
(63) Continuation of application No. 12/407,491, filed on Mar. 19, 2009.

(51) Int. Cl. C12Q 1/04; C12M 3/00

(52) U.S. Cl. 435/34; 435/288.7

(57) ABSTRACT

There is provided a detection device that comprises a light source, a light sensor and an at least partially light-transmitting element that supports growth of at least one analyte selected from among fungi and bacteria. The light source, the at least partially light-transmitting element and the light sensor are positioned relative to one another and oriented relative to one another such that when the light source is illuminated, at least some of the light emitted from the light source passes through the at least partially light-transmitting element and is received by the light sensor. There is also provided a method of detecting an analyte, comprising illuminating a light source to emit light, and detecting an intensity of light that passes through an at least partially light-transmitting device that comprises an at least partially light-transmitting element that supports growth of at least one analyte selected from among fungi and bacteria.

### CROSS-REFERENCE TO RELATED APPLICATIONS

This application is a continuation of U.S. patent application Ser. No. 12/407,491, filed Mar. 19, 2009, the entirety of which is incorporated herein by reference.

### FIELD OF THE INVENTIVE SUBJECT MATTER

The present inventive subject matter relates to a detection device and a method for detecting one or more analytes selected from among fungi and bacteria.

### BACKGROUND

There is an ongoing need for devices and methods which more effectively detect various types of fungi and bacteria.

Various types of fungi and bacteria, for example, can cause health issues and/or product spoilage issues. It is well known that mold, a species of fungus, reproduces by releasing spores into the air, and that the spores that land on moist objects may grow. Also well known are the negative effects on human health that mold can have, and the need to remove materials, especially porous materials such as wallboard, on which mold has been found. The potential negative effects on human health that bacteria can have are also well known.

While existing devices and methods for detecting fungus and/or bacteria are useful to some degree, there is an ongoing need for devices and methods (for detecting specific analytes selected from among fungi and bacteria) that are simpler, more reliable, and/or more sensitive.

### BRIEF SUMMARY OF THE INVENTIVE SUBJECT MATTER

In accordance with a first aspect of the present inventive subject matter, there is provided a detection device comprising an at least partially light-transmitting element that supports growth of an analyte selected from among fungi and bacteria, a light source and a light sensor. In this aspect of the present inventive subject matter, the light source, the at least partially light-transmitting element and the light sensor are positioned relative to one another and oriented relative to one another such that when the light source is illuminated, at least some of the light emitted from the light source passes through the at least partially light-transmitting element and is received by the light sensor. If the at least one fungus and/or at least one bacterium for which detection is desired is present in at least a threshold amount, the at least one fungus and/or at least one bacterium collects on the at least partially light-transmitting element over time and eventually attenuates the intensity of light from the light source that is received by the light sensor.

In accordance with a second aspect of the present inventive subject matter, there is provided a method of detecting at least one analyte selected from among fungi and bacteria, comprising illuminating a light source to emit light, and detecting an intensity of light that passes through an at least partially light-transmitting device comprising an at least partially light-transmitting element that supports growth of the at least one analyte.

In some embodiments according to the present inventive subject matter, the analyte is a particular type of mold or is one of a particular group of mold types, and if one or more of such types of mold is/are present in a substantial amount (i.e., an amount which is at a level that is high enough that detection is desired), the mold grows on the at least partially light-transmitting element.

In some embodiments according to the present inventive subject matter, the analyte is a particular bacterium or one of a particular group of bacteria types, and if such bacterium (or one or more of such types of bacteria) is/are present in a substantial amount, the bacterium (or bacteria) grows on the at least partially light-transmitting element.

In some embodiments according to the present inventive subject matter, the detection device (or the at least partially light-transmitting device) further comprises an at least partially light-transmitting substrate having at least a first surface on which the at least partially light-transmitting element is mounted. In some of such embodiments, the at least partially light-transmitting element is directly mounted on the first surface of the substrate. In some of such embodiments, when the light source is illuminated, at least some of the light emitted from the light source passes through the substrate, then through the at least partially light-transmitting element, and is then received by the light sensor. In some of such embodiments, when the light source is illuminated, at least some of the light emitted from the light source passes through the at least partially light-transmitting element, then through the substrate, and is then received by the light sensor.

In some embodiments according to the present inventive subject matter, the detection device (or the at least partially light-transmitting device) further comprises an at least partially light-transmitting substrate having at least a first surface and a second surface, a first portion of the at least partially light-transmitting element being mounted on the first surface, a second portion of the at least partially light-transmitting element being mounted on the second surface.

In some embodiments according to the present inventive subject matter, the at least partially light-transmitting element comprises at least one cellulose-based material.

In some embodiments according to the present inventive subject matter, the at least partially light-transmitting element is a thin film having an aspect ratio of at least 25.

In some embodiments according to the present inventive subject matter, the detection device further comprises at least one piezoelectric element and at least first and second electrodes positioned on the piezoelectric element and being spaced from each other, whereby current can be applied through the electrodes to cause at least a first region of the piezoelectric element to vibrate. In such embodiments, the piezoelectric element can be caused to vibrate at its natural (or resonant) frequency, by applying electricity to the electrodes, whereby mass change (i.e., the mass deposited on the detection device) can be detected. In some of such embodiments, the piezoelectric element is at least partially light-transmitting, and when the light source is illuminated, at least some of the light emitted from the light source passes through the at least partially light-transmitting element and the piezoelectric element before being received by the light sensor.

In some embodiments according to the first aspect of the present inventive subject matter, the detection device further comprises at least one fluid movement device that, when active, causes fluid to move relative to an exposed surface of the at least partially light-transmitting element.

In some embodiments according to the second aspect of the present inventive subject matter, the method further comprises moving fluid relative to an exposed surface of the at least partially light-transmitting element.

In some embodiments according to the second aspect of the present inventive subject matter, the method further comprises comparing the detected intensity of light that passes through the at least partially light-transmitting device with a threshold intensity, and activating an alarm when the detected intensity falls below the threshold intensity.

In some embodiments according to the second aspect of the present inventive subject matter, the method further comprises comparing the detected intensity of light that passes through the at least partially light-transmitting device with an initial intensity, and activating an alarm when the detected intensity falls below a specific fraction of the initial intensity.

The inventive subject matter may be more fully understood with reference to the accompanying drawings and the following detailed description of the inventive subject matter.

### BRIEF DESCRIPTION OF THE DRAWING FIGURES

FIG. 1 depicts a detection device 10 in accordance with a first embodiment of the present inventive subject matter.

FIG. 2 depicts a detection device 20 in accordance with a second embodiment of the present inventive subject matter.

FIG. 3 depicts a detection device 30 in accordance with a third embodiment of the present inventive subject matter.

FIG. 4 depicts a detection device 40 in accordance with a fourth embodiment of the present inventive subject matter.

FIG. 5 depicts a detection device 50 in accordance with a fifth embodiment of the present inventive subject matter.

FIG. 6 depicts a detection device 60 in accordance with a sixth embodiment of the present inventive subject matter.

FIG. 7 depicts a detection device 70 in accordance with a seventh embodiment of the present inventive subject matter.

FIG. 8 depicts a detection device 80 in accordance with an eighth, embodiment of the present inventive subject matter.

### DETAILED DESCRIPTION OF THE INVENTIVE SUBJECT MATTER

When an element is referred to herein as being “mounted on” another element, it can be directly on the other element, or intervening elements may also be present. In contrast, when an element is referred to herein as being “directly mounted on” another element, there are no intervening elements present. In addition, a statement that a first element is “on” a second element is synonymous with a statement that the second element is “on” the first element.

Although the terms “first”, “second”, etc. may be used herein to describe various elements, components, regions, layers, sections and/or parameters, these elements, components, regions, layers, sections and/or parameters should not be limited by these terms. These terms are only used to distinguish one element, component, region, layer or section from another region, layer or section. Thus, a first element, component, region, layer or section discussed below could be termed a second element, component, region, layer or section without departing from the teachings of the present inventive subject matter.

The expression “at least partially light-transmitting”, as used herein, means that the structure which is characterized as being at least partially light-transmitting allows passage of at least some electromagnetic radiation, and in some cases at least 70% of at least one wavelength of light (e.g., at least one wavelength to which the light sensor is sensitive), and in some cases at least 80% of at least one wavelength of light, and in some cases at least 90% of at least one wavelength of light, and in some cases substantially all of at least one wavelength of light.

As noted above, a first aspect of the present inventive subject matter is directed to a detection device comprising a light source, a light sensor, and an at least partially light-transmitting element that supports growth of at least one analyte selected from among fungi and bacteria.

The at least partially light-transmitting element (that supports growth of at least one analyte selected from among fungi and bacteria) is made of one or more partially light-transmitting materials that support growth of at least one analyte selected from among fungi and bacteria.

Persons of skill in the art are familiar with a variety of at least partially light-transmitting materials that support growth of particular analytes selected from among fungi and bacteria. For example, degraded cellulose is a medium that is particularly suitable for growing various types of molds, like black mold (Stachybotrys). The selection of the at least partially light-transmitting material is based on the analyte for which detection is sought. For example, if there is a need to detect whether a particular analyte (or any of a group of analytes) is present, the at least partially light-transmitting material can be selected so as to minimize false positive reactions resulting from the analyte supporting growth of materials for which detection is not sought, and/or plural at least partially light-transmitting materials can be used where more of the at least partially light-transmitting materials support growth of the analyte(s) than support growth of materials that are not analytes.

The at least partially light-transmitting element can be of any desired shape and size, and persons of skill in the art can readily envision a wide variety of shapes and sizes of which the at least partially light-transmitting element can be.

As noted above, in some embodiments, the at least partially light-transmitting element is a thin film having an aspect ratio of at least 25. The expression “aspect ratio” is used herein in accordance with its well known meaning, i.e., a ratio of one dimension of an article divided by another dimension of the article. An example of an at least partially light-transmitting element having an aspect ratio of at least 25 is an at least partially light-transmitting element having a length, a width and a thickness (or height), where the width (or the length, or both the width and the length) is 25 times (or 30 times, or 35 times, etc.) the thickness.

Where the at least partially light-transmitting material cannot stay in place by itself (e.g., where it is a viscous liquid, e.g., “butter-like” at ambient temperature) structure can be provided that holds the at least partially light-transmitting material in place. For instance, if the at least partially light-transmitting material is “butter-like”, it can be spread on a surface of an at least partially light-transmitting substrate, discussed in more detail below.

As noted above, some embodiments of the detection device according to the present inventive subject matter further comprise an at least partially light-transmitting substrate. Persons of skill in the art are familiar with many materials (e.g., glass or plastic) that can be used to make an at least partially light-transmitting substrate, and any of such materials can be used, as desired.

Persons of skill in the art are familiar a variety of light sources and light sensors that can be used according to the present inventive subject matter to detect reduction of intensity in the light received as a result of build-up of analyte on the at least partially light-transmitting material(s). The light emitted can be any desired kind of electromagnetic radiation, such as infrared light, visible light, ultraviolet light, etc. Persons of skill in the art are familiar with, and have ready access to, a variety of light sources that emit such light, e.g., light emitting diodes (LEDs), and any of such light sources can be employed in accordance with the present inventive subject matter. Similarly, persons of skill in the art are familiar with, and have ready access to, a variety of light sensors, e.g., solid state photo detectors, and any of such light sensors can be employed in accordance with the present inventive subject matter.

Directing light such that at least a portion of the light enters the detection device can be carried out in any of a variety of ways which are well known to those skilled in the optical monitoring art. In some embodiments, light from the source can be affected, e.g., by positioning a monochromator somewhere between the source and the detector. Alternatively, if desired, broadband light sources can be used.

As noted above, in some embodiments, the detection device further comprises at least one fluid movement device which, when active, causes fluid to move relative to an exposed surface of the at least partially light-transmitting element. Persons of skill in the art are familiar with a wide variety of fluid movement devices, e.g., fans, any of which can be used in the present inventive subject matter, if desired.

In embodiments that include one or more fluid movement device(s), the one or more fluid movement device(s), can be oriented in any desired manner relative to the at least partially light-transmitting element. In some embodiments, at least one fluid movement device is positioned generally downstream (i.e., relative to the direction in which fluid is caused to move) from the at least partially light-transmitting element, and/or such that the fluid movement device sucks fluid across the at least partially light-transmitting element (as opposed to pushing fluid across the at least partially light-transmitting element, as is done in other embodiments). In some embodiments in which fluid is sucked across the at least, partially light-transmitting element, by sucking fluid across at least partially light-transmitting element, better sampling of the fluid in the region being monitored is obtained, e.g., more ambient air in a room or space is passed across the substrate (for example, in the case of monitoring for mold, there is a greater chance that spores in the region will get to the at least partially light-transmitting element).

If desired, the detection device can include one or more additional at least partially light-transmitting structures, e.g., layers, which can be positioned in any relation to the other components of the device (e.g., on a surface of the at least partially light-transmitting element, between the at least partially light-transmitting element and an at least partially light-transmitting substrate (if provided), etc.

As noted above, a second aspect of the present inventive subject matter is directed to a method of detecting an analyte, comprising illuminating a light source to emit light, and detecting an intensity of light that passes through an at least partially light-transmitting device comprising an at least partially light-transmitting element that supports growth of an analyte. The descriptions of light sources, suitable at least partially light-transmitting devices, suitable at least partially light-transmitting elements and analytes relating to the first aspect of the present inventive subject matter (as set forth above) are also applicable to the second aspect of the present inventive subject matter.

The light emitted by the light source and received by the light sensor can be in the form of a continuous beam, a flashing light or intermittent flashes of light (separated by uniform or non-uniform time spans).

As noted above, in some embodiments according to the second aspect of the present inventive subject matter, the at least partially light-transmitting device further comprises an at least partially light-transmitting substrate. The description of at least partially light-transmitting substrates relating to the first aspect of the present inventive subject matter (as set forth above) are also applicable to the second aspect of the present inventive subject matter.

As noted above, some embodiments according to the second aspect of the present inventive subject matter further comprise comparing the detected intensity of light that passes through the at least partially light-transmitting device with a threshold intensity and/or with an initial intensity, and activating an alarm when the detected intensity falls below the threshold intensity and/or below a specific fraction of the initial intensity. The comparison can be made in any suitable way, including using a processor. Persons of skill in the art are familiar with a wide variety of alarms, e.g., an audible alarm or an RF signal, and any desired alarm (or alarms) can be used.

In accordance with the present inventive subject matter, when conditions (e.g., temperature and humidity) are suitable for the analyte (or one or more of the analytes) being monitored, e.g., one or more types of mold, the analyte (e.g., mold spores) can begin to grow on the at least partially light-transmitting element, which will cause the at least partially light-transmitting element to become more opaque, and the light passing through will as a result be diminished. The loss in intensity can be used to trigger an alarm. The detection devices and/or methods in accordance with the present inventive subject matter can be used in any location for any desired application, e.g., in homes, businesses, institutions and vessels where mold buildup (or the potential for mold buildup to occur) is a concern. The monitoring can be conducted real-time (or delayed, if desired), and can be conducted continuously (twenty-four hours a day, 365 days a year, etc.), or can be switched on and off as desired.

As noted above, in some embodiments according to the present inventive subject matter, the detection device further comprises at least one piezoelectric element and at least first and second electrodes. Persons of skill in the art are familiar with a variety of materials that can be used for the piezoelectric element and for the electrodes. Persons of skill in the art are also familiar with a variety of at least partially light-transmitting materials that can be used to make at least partially light-transmitting piezoelectric elements.

In addition, persons of skill in the art are familiar with piezoelectric microbalances, i.e., devices in which a piezoelectric element is vibrated at its natural (or resonant) frequency (which changes as a result of small changes in the mass of the device, e.g., as small thicknesses of materials are deposited on a surface of the microbalance) to detect changes in mass.

For example, microbalances, piezoelectric elements and at least partially light-transmitting piezoelectric elements are described in:
(1) U.S. Pat. No. 6,820,485 (inventor: Scott Grimshaw), the entirety of which is hereby incorporated by reference;
(2) U.S. Pat. No. 7,176,474 (inventor: Scott Grimshaw), the entirety of which is hereby incorporated by reference; and
(3) U.S. Pat. No. 7,275,436 (inventor: Scott Grimshaw), the entirety of which is hereby incorporated by reference.

In some embodiments according to the present inventive subject matter that include one or more piezoelectric elements,

the piezoelectric element has at least first and second surfaces opposite one another, and

the first and second surfaces of the piezoelectric element are independently substantially convex, substantially concave or substantially flat.

In some embodiments according to the present inventive subject matter that include one or more piezoelectric elements, at least a portion of at least the first electrode is at least partially light-transmitting. In some such embodiments, at least a portion of at least the second electrode is at least partially light-transmitting.

In embodiments that include one or more piezoelectric elements, the piezoelectric element(s) can generally be made of any piezoelectric material, a number of which are well-known to those skilled in the art. Representative examples of suitable piezoelectric materials include elements made of quartz (e.g., polished quartz) or gallium phosphate. In some embodiments that include a piezoelectric material comprising quartz crystal, the crystal is a singly rotated cut (e.g., an AT-cut crystal) or a doubly rotated cut (e.g., an IT-cut crystal or an SC-cut crystal), such crystal cuts being well-known to those of skill in the art. In embodiments that include one or more piezoelectric elements, the piezoelectric element can generally be of any suitable shape, so long as it can be caused to vibrate by applying electric current between electrodes formed on a surface thereof. In some embodiments of the present inventive subject matter that include one or more piezoelectric elements, the piezoelectric element is substantially plano-convex or has opposite surfaces which are substantially flat and parallel, a representative shape being substantially cylindrical with the axial dimension being much smaller than the radial dimension. In some embodiments that include one or more piezoelectric elements, the edges of the piezoelectric element(s) can be beveled, as is well-known in the art.

As noted above, in some embodiments that include one or more piezoelectric elements, at least part of the piezoelectric element is at least partially light-transmitting. In such embodiments, any desired translucent piezoelectric material can be employed in accordance with the present inventive subject matter, and persons of skill in the art are aware of, and have ready access to, a wide variety of such translucent piezoelectric materials.

In some embodiments that include one or more piezoelectric elements, at least part of the piezoelectric element is reflective. In such embodiments, any desired reflective piezoelectric material can be employed in accordance with the present inventive subject matter, and persons of skill in the art are aware of, and have ready access to, a wide variety of such reflective piezoelectric materials.

In some embodiments that include one or more piezoelectric elements, electrodes can be provided which are of any shape and made of any material such that they can be applied to a surface of the piezoelectric element and can be used to apply current to the piezoelectric element so as to cause the piezoelectric element to vibrate. A variety of electrode shapes and materials are suitable for this purpose, and such materials, e.g., gold, copper and other metals, are well-known to those skilled in the art. For example, in some embodiments according to the present inventive subject matter, parallel field excitation is a well-known technique for causing a piezoelectric element to vibrate, and persons of skill in the art are familiar with a wide variety of structures (piezoelectric elements and electrodes) which can be used to construct chips which will exhibit parallel field excitation of the piezoelectric element when appropriate current is applied to the electrodes. Similarly, persons of skill in the art are familiar with surface acoustic wave (SAW) arrangements, flexural plate wave (FPM) arrangements, and a number of other piezoelectric element-vibrating electrode arrangements that can be constructed. In some embodiments of the present inventive subject matter, electrode/piezoelectric element combinations are employed in which the electrodes do not cover a center region in the crystal.

Those skilled in the art are familiar with providing a suitable power supply for applying a voltage between electrodes of piezoelectric elements as described above in connection with some embodiments of the present inventive subject matter. In addition, persons skilled in the thin film monitoring art are familiar with resonant frequency detectors which can detect a resonant frequency of vibration of detection devices that include piezoelectric elements. Any of such power supplies and any of such resonant frequency detectors can be employed in embodiments in accordance with the present inventive subject matter that include one or more piezoelectric elements.

The detection of resonant frequency can be carried out in any suitable way, including those well known to those of skill in the thin film monitoring art. In carrying out the detection of resonant frequency, the frequency of vibration of the piezoelectric element is sensed using any suitable device. For example, skilled artisans are familiar with microprocessors which can be readily set up to read frequency of vibration of a piezoelectric element. In addition, skilled artisans are familiar with setting up microprocessors to perform such conversions. A variety of algorithms for performing such calculations are well-known to those of skill in the art (see, e.g., Chih-shun Lu, “Mass determination with piezoelectric quartz crystal resonators,” J. Vac. Sci. Technol., Vol. 12, No. 1, (January/February 1975), the entirety of which is hereby incorporated by reference). Corrections can be made, if necessary or desired, to account for acoustic impedance, as is well known in the art. In addition, well known electronics and shielding can be preferably employed in order to eliminate radio frequency interference and voltage variations.

As noted above, in some embodiments in accordance with the present inventive subject matter, there is further provided at least one color change agent positioned on at least a first region of the at least partially light-transmitting element.

In some embodiments according to the present inventive subject matter, the color change agent reacts with the analyte or some material produced by the analyte, and the products of such reaction produce a change in the color of at least some of the light that passes through the at least partially light-transmitting device.

In some embodiments according to the present inventive subject matter, the color change agent reacts with waste products from mold growth, and the products of such reaction produce a change in the color of at least some of the light that passes through the at least partially light-transmitting device. Persons of skill in the art can envision a variety of ways in which the products of such reaction can produce such color change, e.g., filtering the light, reflecting some light, absorbing and re-emitting light of a different wavelength or wavelengths, etc.

In some embodiments according to the present inventive subject matter, the analyte is excretion from at least a first type of mold, and the color change agent comprises at least one material which will exhibit altered light wavelength absorption if it is contacted with the excretion at a concentration which is at least a first mold excretion threshold concentration for the color change agent. In some embodiments according to the present inventive subject matter, the detection device further comprises food for the analyte, e.g., food for one or more types of mold to increase the production of waste products from the analyte.

As noted above, in some embodiments, a color change agent is provided that comprises at least one material which will exhibit altered light wavelength absorption if it is contacted with a material comprising a first analyte at a concentration which is at least a first analyte threshold concentration. The expression “altered light wavelength absorption”, as used herein, means that the group of wavelengths of light which are absorbed by the first color change agent are altered, i.e., there is at least one light wavelength (1) which is absorbed by the first color change agent after contact with the first analyte and is not absorbed by the first color change agent before contact with the first analyte, or (2) which is absorbed by the first color change agent before contact with the first analyte but is not absorbed by the first color change agent after contact with the first analyte, and/or in which the ratio of light absorbed is altered depending on the presence and/or concentration of analyte (or product of analyte).

Persons of skill in the art are familiar with, and have ready access to, a wide variety of color change agents which exhibit altered light wavelength absorption is contacted with particular analytes, and any such color change agents can be employed according to the present inventive subject matter for detecting such corresponding analytes. In addition, persons of skill in the art are familiar with methods for directing illumination which includes light of one or more wavelength which is absorbed by the color change agent before or after (but not both) exposure to the corresponding analyte and detecting whether such light is absorbed by the color change agent in order to determine whether the color change agent has been exposed to the analyte.

Color change agents are readily available for detecting excretion by particular forms of mold at a concentration which is at least a first mold excretion threshold concentration for the color change agent. Likewise, a wide variety of other color change agents are readily available. In the case of detection devices according to the present inventive subject matter in which the color change agent comprises one or more agent for detecting excretion by one or more particular forms of analyte, the detection device can further include one or more substance that the analyte(s) of interest consume (e.g., “mold food”) in order to facilitate the production of such excretion by the analyte(s) of interest.

Persons of skill in the art are likewise familiar with and have ready access to a wide variety of color detectors for determining whether light which has passed (at least once) through a color change agent contains light of one or more particular wavelength. Any such color detectors can be employed according to the present inventive subject matter. Such devices include at least one detection area, and they output a signal which is indicative of whether illumination incident upon the detection area includes light of the one or more wavelength of interest (i.e., “detection wavelength”).

In some embodiments according to the present inventive subject matter that include a color change agent, a region of the color change agent which includes a first surface of the color change agent has a first refractive index,

a region which abuts the first surface of the color change agent has a second refractive index, and

the first refractive index, the second refractive index and the orientation of the beam of light relative to the first surface of the color change agent are such that at least a portion of the beam of light is internally reflected at the first surface of the color change agent after passing through a portion of the color change agent.

In some embodiments of the present inventive subject matter that include a color change agent, the refractive index of a surface of the color change agent differs from that of another material or fluid (liquid or gas) to such an extent that a beam of light can be directed toward the color change agent at such an orientation that it is at least partially internally reflected. Persons of skill in the art can readily determine the refractive indexes of color change agents and abutting materials or fluids in order to determine whether such internal reflection is possible and, if so, the precise orientation of the light source that will provide for such internal reflection.

In some embodiments in accordance with the present inventive subject matter, two measurement techniques are combined in a single device, i.e., piezoelectric crystal vibration monitoring and color change detection are performed at the same location or near the same location. In such embodiments, several advantages are provided: 1) there is no need to set up and purchase two different types of instrumentation in order to accurately monitor coating processes, 2) errors in correlating the two different measurement techniques, due to different geometrical placement of the two devices, are eliminated since one device does both functions, 3) the simultaneous measurement of two features, namely, changes in mass (as measured by piezoelectric crystal vibration monitoring) and absorption properties (as measured optically), reveals previously unavailable information regarding real-time process interactions, such as the effect of mass increase on the optical characteristics (color absorption, etc.), and/or 4) the device in essence is its own backup in the event of failure of the sensor in one measurement technique (i.e., if the crystal in a piezoelectric crystal vibration monitor stopped vibrating, the optical measurement technique is still enabled, and vice versa). In such embodiments, the accuracy of analyte detection can be improved by performing both piezoelectric crystal vibration monitoring and color absorption monitoring at the same location or near the same location. In some embodiments in accordance with the present inventive subject matter, there are provided systems and methods which make it possible to perform both piezoelectric crystal vibration monitoring and color absorption monitoring at the same location (or near the same location). The expression “near the same location,” as used herein, means that the respective locations are spaced from each other by at most 10% of the longest dimension of the surface on which they are located (e.g., on a circular surface having a diameter of 1 cm, the respective locations are spaced from each other by not more than 0.1 cm). In such embodiments, correlation errors which would otherwise be introduced as a result of piezoelectric crystal vibration monitoring and color absorption monitoring being performed at different positions can be avoided.

In some embodiments according to the present inventive subject matter, the detection device comprises at least one reflective element, such that at least a portion of a beam of light reflected at least once, in addition to being passed through the at least partially light-transmitting element (and optionally also passed through a substrate and/or a color change agent). In such embodiments, the reflection can occur at any desired stage, i.e., before or after passing through the at least partially light-transmitting element, etc.

FIG. 1 depicts a detection device 10 in accordance with a first embodiment of the present inventive subject matter. The detection device 10 comprises a substantially transparent element 12 (i.e., an element 12 that is at least partially light-transmitting), a light source 13 and a light sensor 14.

As shown in FIG. 1, the light source 13, the substantially transparent element 12 and the light sensor 14 are positioned relative to one another and oriented relative to one another such that when the light source 13 is illuminated, at least some of the light emitted from the light source 13 passes through the substantially transparent element 12 and is then received by the light sensor 14.

FIG. 2 depicts a detection device 20 in accordance with a second embodiment of the present inventive subject matter. The detection device 20 comprises a substantially transparent substrate 21 (i.e., a substrate 21 that is at least partially light-transmitting), a substantially transparent element 22 (i.e., an element 22 that is at least partially light-transmitting), a light source 23 and a light sensor 24.

The substantially transparent substrate 21 is a clear glass slide having a first surface 25 and a second surface 26. The substantially transparent element 22 is made by coating on the substantially transparent substrate 21 Collodion (which comprises nitrocellulose in an organic solvent), and then drying the Collodion to remove most of the organic solvent, leaving a clear, dry, substantially transparent element 22 comprising nitrocellulose. The resulting substantially transparent element supports growth of certain molds, such as black mold. The substantially transparent element 22 is directly mounted on the first surface 25 of the substantially transparent substrate 21. The light source 23 is an LED, and the light sensor 24 is a solid state photo detector.

As shown in FIG. 2, the light source 23, the substantially transparent substrate 21, the substantially transparent element 22 and the light sensor 24 are positioned relative to one another and oriented relative to one another such that when the light source 23 is illuminated, at least some of the light emitted from the light source 23 passes through the substantially transparent substrate 21, then through the substantially transparent element 22 and is then received by the light sensor 24.

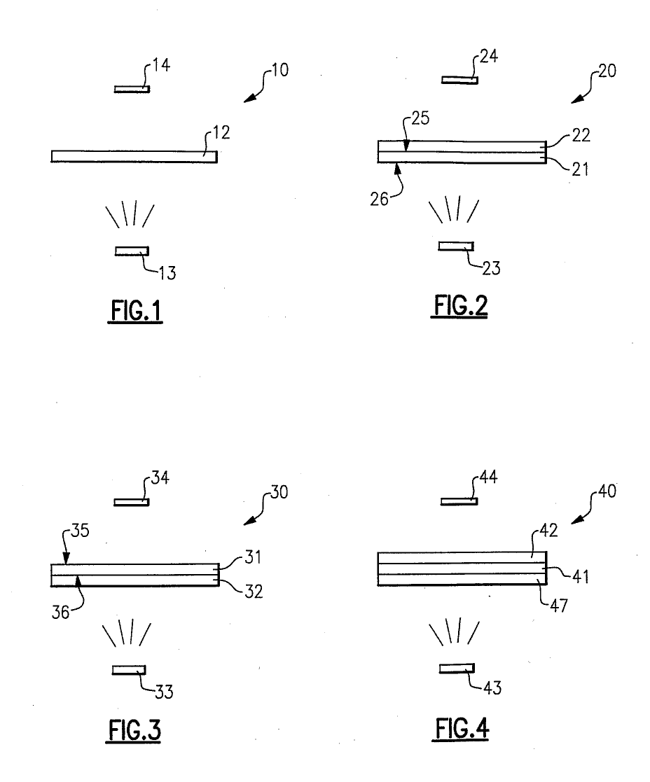

FIG. 3 depicts a detection device 30 in accordance with a third embodiment of the present inventive subject matter. The detection device 30 comprises a substantially transparent substrate 31 (i.e., a substrate 31 that is at least partially light-transmitting), a substantially transparent element 32 (i.e., an element 32 that is at least partially light-transmitting), a light source 33 and a light sensor 34. The substantially transparent substrate 31 is a clear glass slide having a first surface 35 and a second surface 36. The substantially transparent element 32 is directly mounted on the second surface 36 of the substantially transparent substrate 31.

As shown in FIG. 3, the light source 33, the substantially transparent substrate 31, the substantially transparent element 32 and the light sensor 34 are positioned relative to one another and oriented relative to one another such that when the light source 33 is illuminated, at least some of the light emitted from the light source 33 passes through the substantially transparent element 32, then through the substantially transparent substrate 31 and is then received by the light sensor 34.

FIG. 4 depicts a detection device 40 in accordance with a fourth embodiment of the present inventive subject matter. The detection device 40 comprises a substantially transparent substrate 41 (i.e., a substrate 41 that is at least partially light-transmitting), a substantially transparent element (i.e., an element that is at least partially light-transmitting) consisting of a first portion 42 and a second portion 47, a light source 43 and a light sensor 44.

As shown in FIG. 4, the light source 43, the substantially transparent substrate 41, the first portion 42 of the substantially transparent element, the second portion 47 of the substantially transparent element and the light sensor 44 are positioned relative to one another and oriented relative to one another such that when the light source 43 is illuminated, at least some of the light emitted from the light source 43 passes through the second portion 47 of the substantially transparent element, then through the substantially transparent substrate 41, then through the first portion 42 of the substantially transparent element and is then received by the light sensor 44.

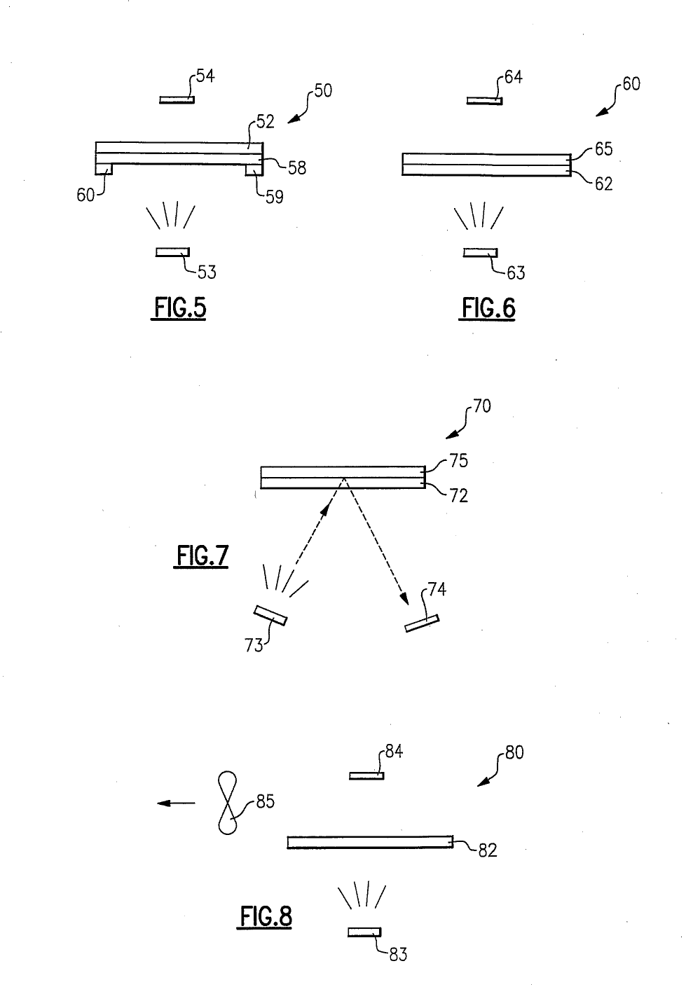

FIG. 5 depicts a detection device 50 in accordance with a fifth embodiment of the present inventive subject matter. The detection device 50 comprises a substantially transparent element 52 (i.e., an element 52 that is at least partially light-transmitting), a substantially transparent piezoelectric element 58 (i.e., a piezoelectric element 58 that is at least partially light-transmitting), a first electrode 59, a second electrode 60, a light source 53 and a light sensor 54.

As shown in FIG. 5, the light source 53, the substantially transparent element 52, the substantially transparent piezoelectric element 58 and the light sensor 54 are positioned relative to one another and oriented relative to one another such that when the light source 53 is illuminated, at least some of the light emitted from the light source 53 passes through the substantially transparent piezoelectric element 58, then passes through the substantially transparent element 52 and is then received by the light sensor 54.

FIG. 6 depicts a detection device 60 in accordance with a sixth embodiment of the present inventive subject matter. The detection device 60 comprises a substantially transparent element 62 (i.e., an element 62 that is at least partially light-transmitting), a light source 63, a light sensor 64 and a color change agent 65 positioned on the substantially transparent element 62.

FIG. 7 depicts a detection device 70 in accordance with a first embodiment of the present inventive subject matter. The detection device 70 comprises a substantially transparent element 72 (i.e., an element 72 that is at least partially light-transmitting), a light source 73, a light sensor 74 and a reflective element 75.

FIG. 8 depicts a detection device 80 in accordance with a first embodiment of the present inventive subject matter. The detection device 80 comprises a substantially transparent element 82 (i.e., an element 82 that is at least partially light-transmitting), a light source 83, a light sensor 84 and a fan 85. The fan 85 sucks air across the substantially transparent element 82, i.e., it causes fluid to move relative to an exposed surface of the at least partially light-transmitting element.

Any two or more structural parts of the detection devices described herein can be integrated. Any structural part of the detection devices described herein can be provided in two or more parts (which can be held together, if necessary). Similarly, any two or more functions can be conducted simultaneously, and/or any function can be conducted in a series of steps.

1. A detection device, comprising:
an at least partially light-transmitting element that supports growth of at least one analyte selected from among fungi and bacteria;
a light source; and
a light sensor,
the light source, the at least partially light-transmitting element and the light sensor being positioned relative to one another and oriented relative to one another such that when the light source is illuminated, at least some of the light emitted from the light source passes through the at least partially light-transmitting element and is received by the light sensor.

2. A detection device as recited in claim 1, wherein the analyte is selected from among molds.

3. A detection device as recited in claim 1, wherein the detection device further comprises an at least partially light-transmitting substrate having at least a first surface on which the at least partially light-transmitting element is mounted.

4. A detection device as recited in claim 3, wherein the at least partially light-transmitting element is directly mounted on the first surface of the substrate.

5. A detection device as recited in claim 3, wherein when the light source is illuminated, at least some of the light emitted from the light source passes through the substrate, then through the at least partially light-transmitting element, and is then received by the light sensor.

6. A detection device as recited in claim 3, wherein when the light source is illuminated, at least some of the light emitted from the light source passes through the at least partially light-transmitting element, then through the substrate, and is then received by the light sensor.

7. A detection device as recited in claim 1, wherein the detection device further comprises an at least partially light-transmitting substrate having at least a first surface and a second surface, a first portion of the at least partially light-transmitting element being mounted on the first surface, a second portion of the at least partially light-transmitting element being mounted on the second surface.

8. A detection device as recited in claim 1, wherein the at least partially light-transmitting element comprises at least one cellulose-based material.

9. A detection device as recited in claim 1, wherein the at least partially light-transmitting element is a thin film having an aspect ratio of at least 25.

10. A detection device as recited in claim 1, wherein the detection device further comprises at least one fluid movement device, the fluid movement device, when active, causing fluid to move relative to an exposed surface of the at least partially light-transmitting element.

11. A detection device as recited in claim 1, wherein the detection device further comprises at least one piezoelectric element and at least first and second electrodes positioned on the piezoelectric element and being spaced from each other, whereby current can be applied through the electrodes to cause at least a first region of the piezoelectric element to vibrate.

12. A detection device as recited in claim 11, wherein:
the piezoelectric element is at least partially light-transmitting; and
when the light source is illuminated, at least some of the light emitted from the light source passes through the at least partially light-transmitting element and the piezoelectric element before being received by the light sensor.

13. A detection device as recited in claim 1, wherein:
the detection device further comprises at least one color change agent positioned on at least a first region of the at least partially light-transmitting element; and
the detection device comprises at least one light color detector which has a detection area and which outputs a signal which is indicative of whether illumination incident upon the detection area includes light of at least one detection wavelength

14. A detection device as recited in claim 13, wherein at least a portion of the color change agent is positioned directly on the first region.

15. A method of detecting an analyte, comprising:
illuminating a light source to emit light; and
detecting an intensity of light that passes through an at least partially light-transmitting device comprising an at least partially light-transmitting element that supports growth of at least one analyte selected from among fungi and bacteria.

16. A method as recited in claim 15, wherein the analyte is selected from among molds.

17. A method as recited in claim 15, wherein the method further comprises moving fluid relative to an exposed surface of the at least partially light-transmitting element.

18. A method as recited in claim 15, wherein the method further comprises comparing the detected intensity of light that passes through the at least partially light-transmitting device with a threshold intensity, and activating an alarm when the detected intensity falls below the threshold intensity.

19. A method as recited in claim 15, wherein the method further comprises comparing the detected intensity of light that passes through the at least partially light-transmitting device with an initial intensity, and activating an alarm when the detected intensity falls below a specific fraction of the initial intensity.

20. A method as recited in claim 15, wherein:
the at least partially light-transmitting device further comprises at least one color change agent positioned on at least a first region of the at least partially light-transmitting element; and
the method further comprises detecting a color of light that contacts a detection area.
